(19) United States
(12) Patent Application Publication
(10) Pub. No.: US 2025/0170310 A1
(43) Pub. Date: May 29, 2025

(54) METHOD OF MANUFACTURING INTRAVASCULAR INDWELLING DEVICE, INTRAVASCULAR-INDWELLING-DEVICE MANUFACTURING HOLDER, AND METHOD OF EVALUATING INTRAVASCULAR INDWELLING DEVICE

(71) Applicants: JSR Corporation, Tokyo (JP); KEIO UNIVERSITY, Tokyo (JP)

(72) Inventors: Takeshi TOKUNAGA, Tokyo (JP); Jitsuro TSUKADA, Tokyo (JP)

(73) Assignees: JSR Corporation, Tokyo (JP); KEIO UNIVERSITY, Tokyo (JP)

(21) Appl. No.: 18/834,453

(22) PCT Filed: Jan. 31, 2023
(86) PCT No.: PCT/JP2023/002985

§ 371 Date: Jul. 30, 2024

(30) Foreign Application Priority Data
Jan. 31, 2022 (JP) 2022-012899

(51) Int. Cl. A61L 31/16; C12N 5/071

(52) U.S. Cl. CPC A61L 31/16; C12N 5/0692; A61L 2400/18; A61L 2430/40

(57) ABSTRACT

A method of manufacturing an intravascular indwelling device uses a cylindrical holder (1) holding the intravascular indwelling device (20) in an internal space, and an outer cylinder (2) having an inner diameter larger than an outer diameter of the holder (1). The holder (1) holding the intravascular indwelling device (20) is accommodated in the outer cylinder (2), and a suspension(S) of cells of interest are supplied into the outer cylinder (2). The cells of interest are cultured by operating the outer cylinder (2) and the holder (1) such that the suspension(S) flows to the intravascular indwelling device (20) while being in contact with the intravascular indwelling device (20).

### TECHNICAL FIELD

The present invention relates to a method of manufacturing an intravascular indwelling device, an intravascular-indwelling-device manufacturing holder, and a method of evaluating the intravascular indwelling device.

Priority is claimed on Japanese Patent Application No. 2022-012899 filed on Jan. 31, 2022, the content of which is incorporated herein by reference.

### BACKGROUND ART

As a countermeasure against re-endothelialization disorders of blood vessels after stent placement, a method of manufacturing a stent that causes vascular endothelial cells to adhere to a surface of the stent in vitro has been proposed. According to the manufacturing method, biocompatibility of the stent is improved by causing vascular endothelial cells to adhere, and the endothelialization ability of the stent is enhanced (refer to, for example, Non-Patent Literature 1).

### CITATION LIST

### Non-Patent Literature

[Non-Patent Literature 1] M. Y. Flugelman, et al., “Genetically Engineered Endothelial Cells Remain Adherent and Viable After Stent Deployment and Exposure to Flow In Vitro”, Circulation Research Vol. 70, No. 2, February 1992, p. 348-354.

### SUMMARY OF INVENTION

### Problem to be Solved by the Invention

In the method of manufacturing the stent described above, there has been cases in which adherence of cells adhered to a surface of the stent is not sufficient, thereby affecting the endothelialization ability of the stent.

One aspect of the invention is to provide a method of manufacturing an intravascular indwelling capable of causing cells to sufficiently adhere to a surface of a stent over a wide range, an intravascular-indwelling-device manufacturing holder, and a method of evaluating the intravascular indwelling device.

### Means to Solve the Problem

The invention includes the following aspects.
[1] A method of manufacturing an intravascular indwelling device having cells of interest adhered thereto, which are at least one of vascular endothelial cells and vascular endothelial progenitor cells, the method includes: using a cylindrical holder holding the intravascular indwelling device in an internal space, and an outer cylinder having an inner diameter larger than an outer diameter of the holder; and accommodating the holder holding the intravascular indwelling device in the outer cylinder, supplying a suspension of the cells of interest into the outer cylinder, culturing the cells of interest by operating the outer cylinder and the holder such that the suspension flows to the intravascular indwelling device while being in contact with the intravascular indwelling device.
[2] The method of manufacturing an intravascular indwelling device according to [1] described above, in which the holder has one or more flow holes allowing the suspension to flow therethrough and are formed to penetrate from an inner circumferential surface to an outer circumferential surface of the holder.
[3] The method of manufacturing an intravascular indwelling device according to [1] or [2] described above, further includes a backflow prevention structure restricting an inflow of a gas into the outer cylinder and is provided at least on one end part of the outer cylinder.
[4] The method of manufacturing an intravascular indwelling device according to any one of [1] to [3] described above, further includes causing the outer cylinder and the holder to rotate around an axis parallel to a central axis of the outer cylinder when the suspension flows.
[5] An intravascular-indwelling-device manufacturing holder used for manufacturing an intravascular indwelling device having cells of interest adhered thereto, which are at least one of vascular endothelial cells and vascular endothelial progenitor cells, the intravascular-indwelling-device manufacturing holder having a cylindrical shape capable of holding the intravascular indwelling device in an internal space, and having one or more flow holes allowing the suspension of the cells of interest to flow therethrough and are formed to penetrate from an inner circumferential surface to an outer circumferential surface.
[6] A method of evaluating an intravascular indwelling device having cells of interest adhered thereto, which are at least one of vascular endothelial cells and vascular endothelial progenitor cells, the method includes ascertaining that a ratio (Q1/Q2) of a lactic acid production amount Q1 to a glucose consumption amount Q2 of the cells of interest is 0.1 or higher when the intravascular indwelling device is cultured in a culture medium at a temperature of 37° C. for 6 hours in a 5% CO₂ environment.
[7] The method of evaluating an intravascular indwelling device according to [6] described above, in which the method ascertains that the ratio (Q1/Q2) is 0.3 or higher.

### Advantageous Effects of Invention

According to one aspect of the invention, it is possible to provide a method of manufacturing an intravascular indwelling capable of causing cells to sufficiently adhere to a surface of a stent over a wide range, an intravascular-indwelling-device manufacturing holder, and a method of evaluating the intravascular indwelling device.

### BRIEF DESCRIPTION OF DRAWINGS

FIG. 1 is a configuration view showing a manufacturing jig used in a method of manufacturing an intravascular indwelling device according to a first embodiment.

FIG. 2 is a cross-sectional view showing the manufacturing jig used in the method of manufacturing the intravascular indwelling device according to the first embodiment.

FIG. 3 is a process view showing the method of manufacturing the intravascular indwelling device according to the first embodiment.

FIG. 4 is a process view showing the method of manufacturing the intravascular indwelling device according to the first embodiment.

FIG. 5 is a process view showing the method of manufacturing the intravascular indwelling device according to the first embodiment.

FIG. 6 is a configuration view showing a culture device used in the method of manufacturing the intravascular indwelling device according to the first embodiment.

FIG. 7 is a configuration view showing an intravascular indwelling device obtained by the method of manufacturing the intravascular indwelling device according to the first embodiment.

FIG. 8 is an enlarged view showing a part of the intravascular indwelling device having cells of interest adhered thereto.

FIG. 9 is a cross-sectional view showing a manufacturing jig used in a method of manufacturing an intravascular indwelling device according to a second embodiment.

FIG. 10 is a cross-sectional view showing the manufacturing jig used in the method of manufacturing the intravascular indwelling device according to the second embodiment.

FIG. 11 is a photograph showing an inner surface of a stent obtained in Example 1.

FIG. 12 is a photograph showing an inner surface of a stent obtained in Example 2.

FIG. 13 is a photograph showing an inner surface of a stent obtained in Comparative Example 1.

FIG. 14 is a photograph showing an inner surface of a stent obtained under condition A.

FIG. 15 is a photograph showing an inner surface of a stent obtained under condition C.

FIG. 16 is a photograph showing an inner surface of a stent obtained under condition D.

### DESCRIPTION OF EMBODIMENTS

### [Method of Manufacturing Intravascular Indwelling Device] (First Embodiment)

### (Manufacturing Jig)

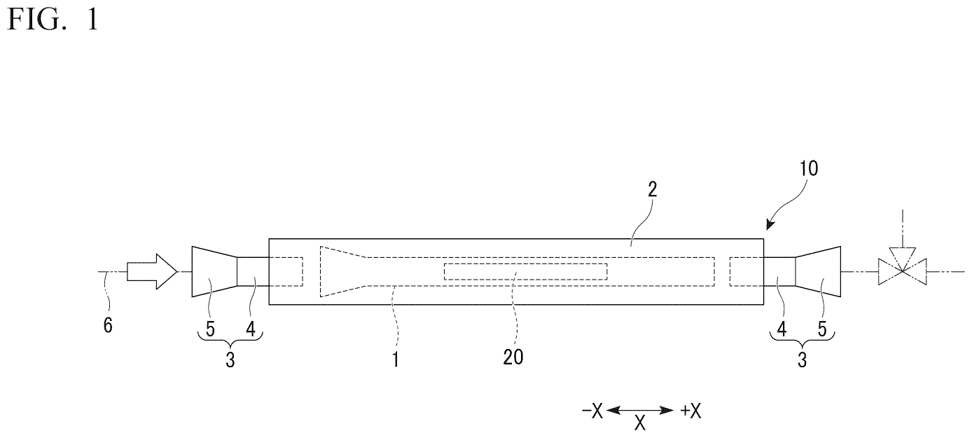

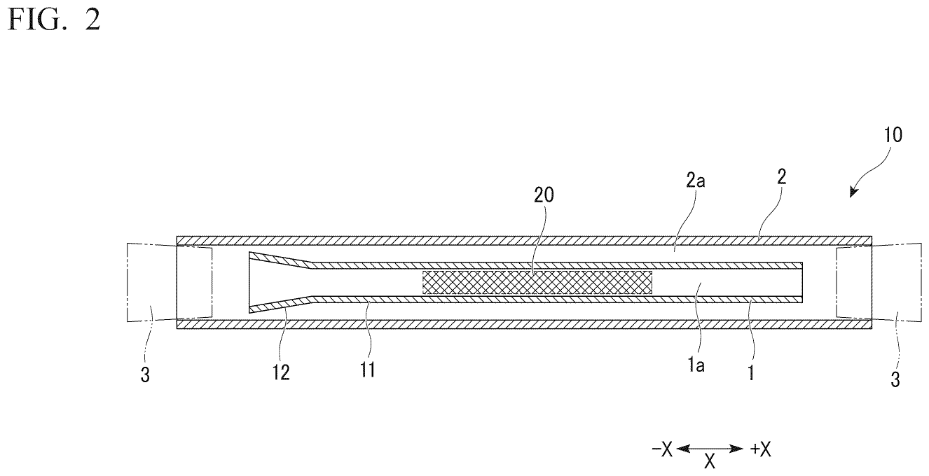

FIG. 1 is a configuration view showing a manufacturing jig 10 used in a method of manufacturing an intravascular indwelling device according to a first embodiment. FIG. 2 is a cross-sectional view showing the manufacturing jig 10.

As shown in FIGS. 1 and 2, the manufacturing jig 10 includes a holder 1, an outer cylinder 2, and two plugs 3. An axial direction of the holder 1 is referred to as an X direction. A direction toward a right end of the holder 1 is referred to as a +X direction. A direction opposite to the +X direction is referred to as a −X direction.

The holder 1 is formed in a cylindrical shape. The holder 1 includes a main portion 11 and an enlarged-diameter portion 12. The main portion 11 has a cylindrical shape. The enlarged-diameter portion 12 extends in the −X direction from one end (an end in the −X direction) of the main portion 11 while an outer diameter becomes larger. The holder 1 is open at both ends.

The holder 1 is formed of, for example, a polymer material. The holder 1 is required to have a sufficient rigidity not to be damaged when it is inserted into a blood vessel using a delivery system or the like. The holder 1 is also required to be difficult for cells of interest to adhere thereto. The holder 1 also needs to withstand a sterilization operation. As materials meeting these requirements, there are polymer materials such as fluorine-based polymers and silicone-based polymers. The holder 1 may also be formed of a metal material.

As shown in FIG. 2, the holder 1 is capable to accommodating and hold a stent 20 in an internal space 1a. An inner diameter of the holder 1 is the same as the outer diameter of the stent 20 in an unexpanded state or is larger than the outer diameter of the stent 20. The stent 20 is inserted into the holder 1 in the unexpanded state. A length direction of the stent 20 held by the holder 1 may be parallel to the axial direction of the holder 1. An outer diameter of the main portion 11 may be, for example, 2.5 mm to 4 mm.

The outer cylinder 2 is formed in a cylindrical or tubular shape. The outer cylinder 2 is capable to accommodating the holder 1 in an internal space 2a. An inner diameter of the outer cylinder 2 is larger than the outer diameter of the holder 1. The inner diameter of the outer cylinder 2 is preferably 1.5 times or more (for example, twice or more) an outer diameter of the main portion 11 of the holder 1. Therefore, it is possible to secure a sufficient space between an inner circumferential surface of the outer cylinder 2 and an outer circumferential surface of the holder 1, and it is possible to improve fluidity of a cell suspension. The inner diameter of the outer cylinder 2 is, for example, four times or less the outer diameter of the main portion 11. Therefore, it is possible to reduce an amount of a cell suspension S used. The inner diameter of the outer cylinder 2 may be, for example, 5 mm to 8 mm.

The holder 1 is inserted into the outer cylinder 2. The axial direction of the holder 1 accommodated in the outer cylinder 2 may be parallel to an axial direction of the outer cylinder 2.

The outer cylinder 2 is formed of a polymer material or the like. As a constituent material of the outer cylinder 2, silicone-based polymers, fluorine-based polymers, or the like may be mentioned. As the outer cylinder 2, a silicone tube is suitable.

The outer cylinder 2 may be formed of a transparent material or an opaque material. If the outer cylinder 2 is formed of a transparent material, since it is possible to visually ascertain the inside of the outer cylinder 2, an operation is facilitated. The outer cylinder 2 may be flexible or non-flexible. The outer cylinder 2 may be formed of a metal material.

As shown in FIG. 1, the plug 3 includes a plug main body 4 and a check valve 5.

The plug main body 4 closes an opening at an end part of the outer cylinder 2 in a liquid-tight manner and prevents leakage of a liquid from the outer cylinder 2. The plug main body 4 is formed of, for example, a polymer material. An insertion hole (not shown in the drawings) through which an instruction tube or a lead-out tube for guiding the cell suspension is inserted is formed in the plug 3.

The check valve 5 is, for example, a check valve with a known structure. The check valve 5 may be, for example, a ball type check valve. The check valve 5 is provided in both the plugs 3. The check valve 5 is an example of a “backflow prevention structure”. The check valve 5 is capable of restricting an inflow of a gas (external air) into the outer cylinder 2. Therefore, in a culture step to be described below, it is possible to maintain a sterile environment, prevent drying, or the like in the manufacturing jig 10.

### (Stent)

As shown in FIG. 2, the stent 20 is formed in a tubular shape. The stent 20 is used for a stent treatment of inserting it into a lumen of a blood vessel to maintain the lumen in an expanded state when an abnormality such as stenosis or occlusion occurs in the lumen. The stent 20 is inserted into the lumen in an unexpanded state, and is expanded and placed inside the lumen.

The stent 20 may be, for example, a tubular body formed of linear objects such as wires. The stent 20 has a structure having, for example, a plurality of annular portions that are formed of linear objects and are connected in the axial direction. A structure of the stent 20 is not particularly limited. The structure of the stent 20 may be, for example, a grid mesh structure, a structure combining a plurality of loops, a spiral structure, or the like.

The stent 20 is preferably a self-expandable stent, but may also be a balloon-expandable stent.

The stent 20 is formed of, for example, a metal material, a polymer material, or the like.

As the metal material, metal materials including nickel, titanium, stainless steel, cobalt, platinum, chromium, tantalum, tungsten, and the like may be mentioned. Suitable metal materials include a nickel-titanium alloy, stainless steel, a cobalt-chromium alloy, a platinum-chromium alloy, and the like.

As the polymer material, examples include polyethylene terephthalate (PET), polybutylene terephthalate (PBT), polycarbonate, polyethylene, polypropylene, polyacetal, polystyrene, polyacrylic acid ester, and the like.

A biodegradable polymer material may also be used as the polymer material. As the biodegradable polymer material, examples include polyhydroxyester-based materials such as polylactic acid, polyglycolic acid, polymalic acid, and copolymers thereof, and polyamide-based materials such as polycaprolactone.

A coating layer of biologically derived substances such as collagen, agarose, and gelatin may be formed on a surface of the stent 20.

The stent 20 may be a drug-eluting stent (DES) having a drug coating layer formed on an outer surface thereof. The drug, which serves as an active ingredient of the drug coating layer, can suppress, for example, migration and proliferation of vascular smooth muscle cells. The drug, which serves as an active ingredient of the drug coating layer, may be, for example, an anticancer drug, an immunosuppressant, an antibiotic, an antirheumatic drug, an antithrombotic drug, and the like. The drug coating layer may be formed only on the outer surface of the stent 20, or may be formed on both the outer surface and an inner surface of the stent 20.

The stent 20 is an example of the “intravascular indwelling device”. The intravascular indwelling device is a structure that can be placed within a blood vessel. In addition to the stent, examples of the intravascular indwelling device include stent grafts, venous filters (for example, inferior vena cava filters), and the like.

### (Method of Manufacturing Intravascular Indwelling Device)

An example of the method of manufacturing the intravascular indwelling device will be described in detail with reference to FIGS. 3 to 6.

### [Step of Preparation]

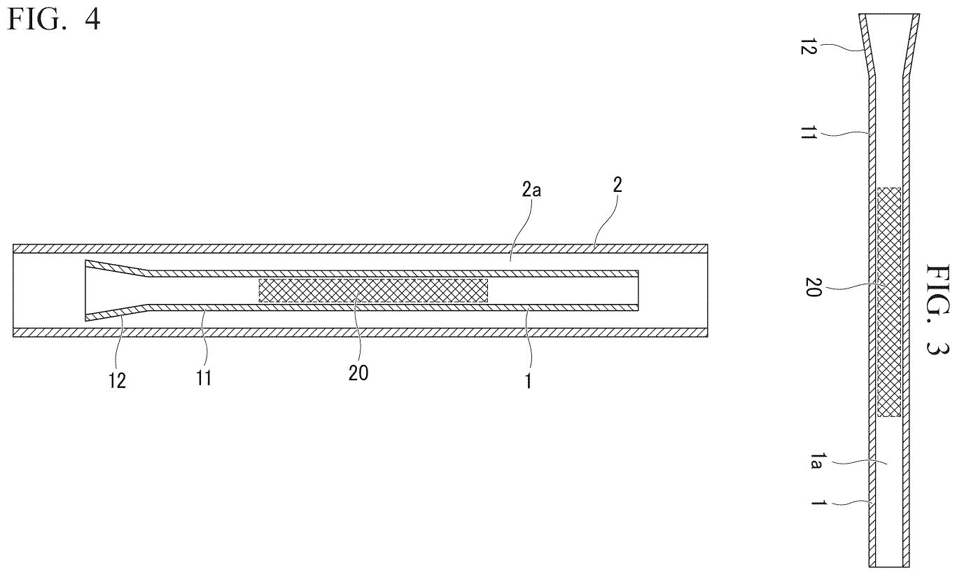

As shown in FIG. 3, the stent 20 is inserted into the holder 1. The holder 1 holds the stent 20 in the internal space 1a. As shown in FIG. 4, the holder 1 is inserted into the outer cylinder 2. The holder 1 is accommodated in the internal space 2a of the outer cylinder 2 in a posture extending in a length direction of the outer cylinder 2.

### [Step of Supplying Cell Suspension]

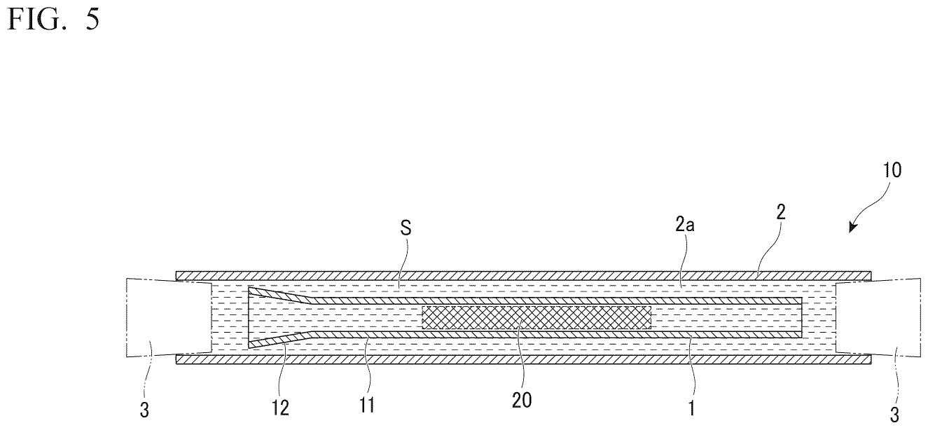

As shown in FIG. 5, the plugs 3 are mounted on the openings at both ends of the outer cylinder 2. The cell suspension S is introduced into the outer cylinder 2 through an introduction tube 6 (refer to FIG. 1).

The cell suspension S is a suspension containing at least one of vascular endothelial cells and vascular endothelial progenitor cells. The vascular endothelial progenitor cells are, for example, late-outgrowth vascular endothelial progenitor cells. At least one of the vascular endothelial cells and the vascular endothelial progenitor cells may be referred to as “cells of interest”. The cells of interest may be either the vascular endothelial cells or the vascular endothelial progenitor cells, or both.

The cell suspension S is supplied to the internal space 2a of the outer cylinder 2. The cell suspension S enters the inside of the holder 1 and comes into contact with the stent 20. In the embodiment, the entire internal space 2a of the outer cylinder 2 is filled with the cell suspension S. The cell suspension S spreads throughout the internal space 1a of the holder 1. The stent 20 is entirely immersed in the cell suspension S.

### [Step of Culture]

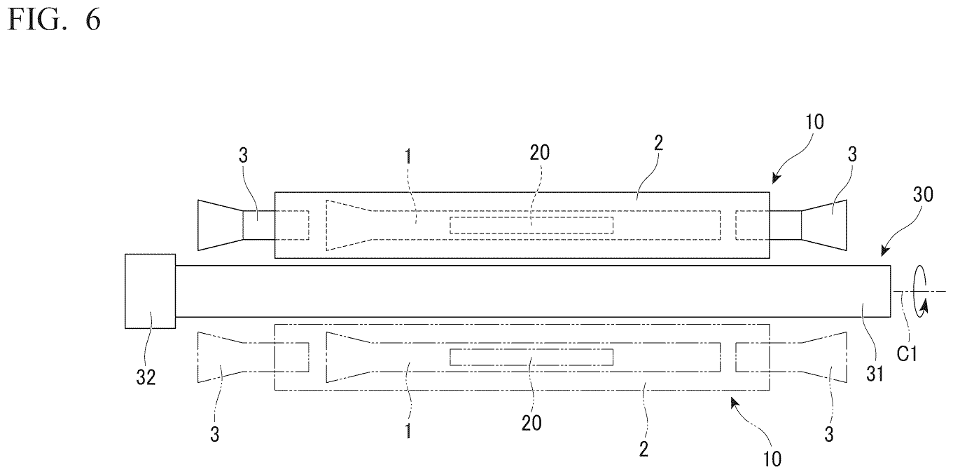

FIG. 6 is a configuration view showing a culture device 30 used in the manufacturing method of the embodiment. The culture device 30 includes a plate-shaped support body 31 and a drive source 32. The support body 31 is rotatable around a central axis C1. The central axis C1 of the support body 31 extends, for example, in a horizontal direction. If the support body 31 is horizontal, the manufacturing jig 10 can be horizontal. Therefore, uniform cell adherence in the axial direction of the stent 20 is possible.

One or more holders (not shown in the drawings) that hold the manufacturing jig 10 are provided on an outer circumferential surface of the support body 31. The drive source 32 is, for example, a motor that rotationally drives the support body 31.

The manufacturing jig 10 filled with the cell suspension S is mounted on the culture device 30. The manufacturing jig 10 is held in the holder in a posture in which a length direction thereof extends in the length direction of the support body 31. The culture device 30 is capable of rotating the manufacturing jig 10 around an axis (central axis C1) parallel to a central axis of the outer cylinder 2.

The number of manufacturing jigs 10 held by the culture device 30 may be one or more. The plurality of manufacturing jigs 10 are mounted, for example, at different positions in a direction around the axis of the support body 31.

The support body 31 is rotated around the axis by the drive source 32. The manufacturing jig 10 rotates around the axis of the support body 31. It is possible to determine conditions for a rotational operation in consideration of parameters related to culturing the cells of interest. The parameters related to the culture include, for example, ecological characteristics of the cells of interest, a shear force applied to the cells of interest by the cell suspension S, flow characteristics of the cell suspension S, specifications (an inner diameter, a volume, and the like) of the outer cylinder 2 and the holder 1, characteristics of the stent, and the like.

It is also possible to determine environmental conditions such as temperature in consideration of the parameters related to culturing the cells of interest.

When the manufacturing jig 10 rotates around the support body 31, the cell suspension S in the outer cylinder 2 flows to the stent 20 while being in contact with the stent 20. It is possible to realize the flow of the cell suspension S to the stent 20 by causing the cell suspension S to flow inside the outer cylinder 2. The flow of the cell suspension S to the stent 20 is also realized by displacing the holder 1 inside the outer cylinder 2. In other words, the flow of the cell suspension S to the stent 20 may be realized by the cell suspension S moving to the stent 20, or may be realized by the stent 20 moving to the cell suspension S.

If the cells of interest are cultured by causing the cell suspension S to flow while being in contact with the stent 20 using the culture device 30, the cells of interest adhere to the surface of the stent 20. The cells of interest adhere to at least the inner surface of the stent 20. It is possible to determine a culture time in consideration of the parameters related to culturing the cells of interest described above.

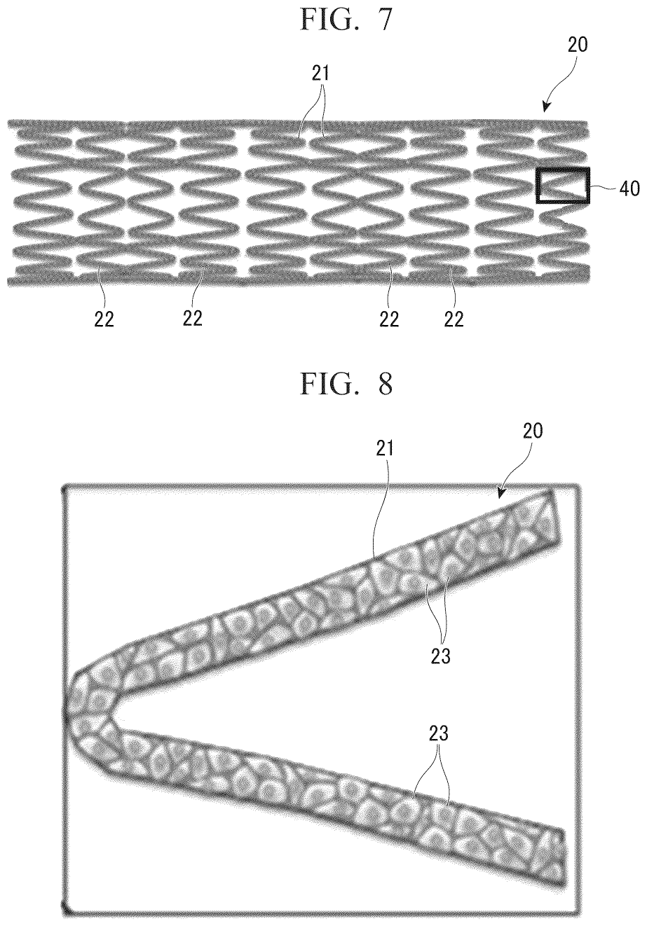

FIG. 7 is a configuration view showing the stent 20 obtained by the manufacturing method of the embodiment. As shown in FIG. 7, the stent 20 is a tubular body having a structure having a plurality of annular portions 22 that are formed of a metal wire 21 and are connected in the axial direction. The annular portion 22 is formed of the zigzag-shaped (SZ-shaped) metal wire 21 having a plurality of folded portions. A sufficient gap is secured between the metal wires 21 constituting the stent 20.

FIG. 8 is an enlarged view showing a part of the stent 20 to which cells of interest have adhered. FIG. 8 is an enlarged view showing a portion surrounded by a rectangular frame 40 shown in FIG. 7.

As shown in FIG. 8, cells of interest 23 are adhered to the surface of the stent 20 by the culture using the culture device 30. In the embodiment, a large number of cells of interest 23 are adhered to the surface of the stent 20 over almost the entire region. Therefore, a distribution of the cells of interest 23 on the surface of the stent 20 is substantially uniform.

An example of use of the stent 20 obtained by the manufacturing method of the embodiment will be described.

The stent 20 in an unexpanded state is inserted into a blood vessel using a delivery system or the like to be disposed in a lumen of a target site (diseased site) of the blood vessel.

If the stent 20 is a self-expandable stent, the stent 20 is autonomously expanded. If the stent 20 is a balloon-expandable stent, the stent 20 is expanded by inflating the balloon. The expanded stent 20 is placed at the target site of the blood vessel. Patency of the blood vessel is secured by the expansion of the stent 20. Therefore, it is possible to realize a satisfactory blood flow at the target site (diseased site) of the blood vessel.

If the cells of interest are vascular endothelial progenitor cells, the vascular endothelial progenitor cells become vascular endothelial cells in the process of the culture using the culture device 30 or after the stent 20 is placed in the blood vessel.

### Effects Obtained by Manufacturing Method of First Embodiment

According to the manufacturing method of the embodiment, since the holder 1 is accommodated in the outer cylinder 2 having a larger diameter than the holder 1, and the cell suspension S is supplied into the outer cylinder 2 to perform culture, it is possible to cause the cell suspension S to flow sufficiently inside the outer cylinder 2, and it is possible to cause the cells of interest to adhere to a wide range on the surface of the stent 20. Therefore, it is possible to promote new vascular endothelial cells to settle and proliferate on the stent 20 placed in the blood vessel. Therefore, the stent 20 can be endothelialized at an early stage.

There is a likelihood that restenosis and reocclusion of a blood vessel after the stent placement may be related to the living body's recognition of the stent as a foreign object. Since the stent 20 obtained by the manufacturing method of the embodiment has the high endothelialization ability, restenosis and reocclusion of a blood vessel are less likely to occur. Therefore, for example, it is possible to suppress late stent thrombosis.

According to the manufacturing method of the embodiment, since the holder 1 is inserted into the outer cylinder 2, even if the inner diameter of the outer cylinder 2 is not so large compared to the outer diameter of the main portion 11 of the holder 1, it is possible to secure sufficient fluidity of the cell suspension S, and it is possible to cause cells of interest to efficiently adhere to the stent 20. Therefore, when the outer cylinder 2 with a small inner diameter is used, it is possible to reduce a required amount of the cell suspension S to be supplied into the outer cylinder 2. If the cells of interest are the patient's own cells (autologous cells) to be treated with the stent, it is often difficult to secure a large amount of cells. Therefore, being able to suppress the required amount of the cell suspension S can be a significant advantage in advancing the stent treatment.

In the manufacturing method of the embodiment, since a holder that has been used in an existing delivery system can be used as the holder 1, it is possible to achieve cost reduction. In the manufacturing method of the embodiment, since the manufacturing jig 10 has a simple structure, it is easy to operate.

In the manufacturing method of the embodiment, it is possible to perform the culture with the plurality of manufacturing jigs 10 held in the culture device 30. Since it is possible to obtain a plurality of stents 20 in one operation, it is possible to increase production efficiency of the stent 20.

Since the culture device 30 can rotate the manufacturing jig 10 around the axis parallel to the central axis of the outer cylinder 2, an inclination of the axis of the outer cylinder 2 can be made constant. Therefore, a flow of the cell suspension S inside the outer cylinder 2 is suppressed. Therefore, even if the cells of interest are susceptible to shear force, it is possible to reduce damage to the cells of interest.

### [Evaluation Method of Intravascular Indwelling Device]

An example of the method of evaluating the intravascular indwelling device according to the embodiment will be described.

It is possible to evaluate the stent 20, to which the cells of interest have adhered (refer to FIGS. 7 and 8), as follows.

The stent 20 to which the cells of interest are adhered is cultured in a culture medium at a temperature of 37° C. for 6 hours in a 5% CO₂ environment. It is ascertained that a ratio (Q1/Q2) of a lactic acid production amount Q1 to a glucose consumption amount Q2 of the cells of interest adhered to the stent 20 is 0.1 or higher. If the ratio (Q1/Q2) is 0.1 or higher, it is possible to evaluate that a sufficient amount of cells of interest are adhered to the stent 20. If an amount of adhered cells of interest is large, it is advantageous for endothelialization of the stent 20.

In the method of evaluating the intravascular indwelling device according to the embodiment, it is possible to ascertain that the ratio (Q1/Q2) is 0.3 or higher. If the ratio (Q1/Q2) is 0.3 or higher, it is possible to evaluate that the amount of cells of interest adhered to the stent 20 is more satisfactory.

Regarding the reason why adherence of cells can be evaluated by the ratio (Q1/Q2), the following conjectures are possible.

If the number of cells is small, an amount of oxygen available per cell is large. Therefore, a glycolytic system including a citric acid cycle works, making it difficult for the lactic acid to be produced relative to the glucose consumption. On the other hand, if the number of cells is large, anaerobic glycolysis becomes active, and the amount of lactic acid production increases relative to the amount of glucose consumption. Therefore, when a sufficient amount of cells adhere to the stent, it is thought that the amount of lactic acid production increases relative to the amount of glucose uptake. Therefore, it is possible to evaluate adherence of cells based on the ratio of the amount lactic acid production and the amount of glucose consumption.

It is possible to measure the amount of lactic acid production and the amount of glucose consumption, for example, by a blood analyzer (Abbott, i-STAT1 Analyzer).

According to this evaluation method, it is possible to appropriately evaluate adherence of cells to the stent 20.

### [Method of Manufacturing Intravascular Indwelling Device] (Second Embodiment)

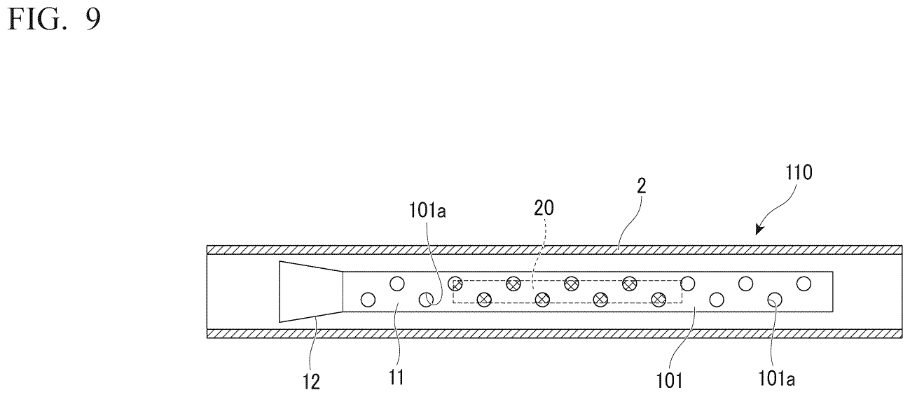

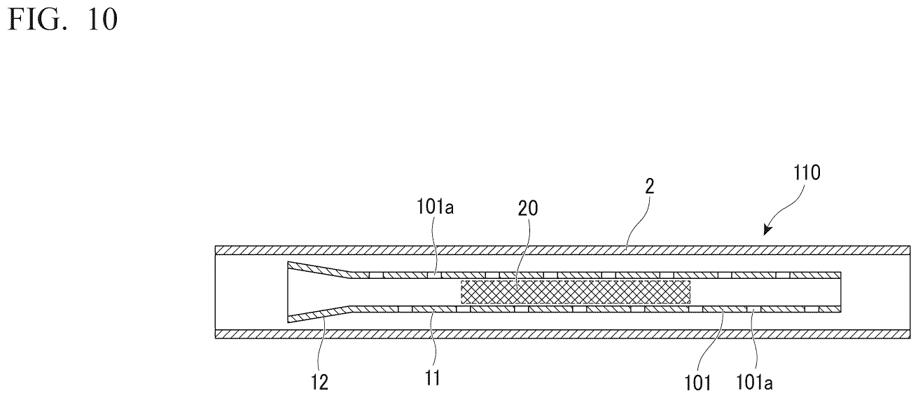

FIG. 9 is a configuration view showing a manufacturing jig 110 used in a method of manufacturing an intravascular indwelling device according to a second embodiment. FIG. 10 is a cross-sectional view showing the manufacturing jig 110.

As shown in FIGS. 9 and 10, the manufacturing jig 110 includes a holder 101, an outer cylinder 2, and a plug 3 (refer to FIG. 1). The manufacturing jig 110 includes the holder 101 instead of the holder 1. The holder 101 may be similar to the holder 1 shown in FIG. 2 except for having the flow hole 101a. The manufacturing jig 110 may have the same configuration as the manufacturing jig 10 shown in FIG. 2 except for the holder 101. The holder 101 is an example of a “intravascular-indwelling-device manufacturing holder”.

The holder 101 has one or more flow holes 101a formed in a main portion 11. It is preferable that the number of flow holes 101a be plural. It is desirable that the plurality of flow holes 101a be formed over a wide range in a length direction and a circumferential direction of the main portion 11. Two or more of the plurality of flow holes 101a are formed at intervals in the length direction of the main portion 11. Two or more of the plurality of flow holes 101a are formed at different positions in the circumferential direction of the main portion 11.

A hole diameter of the flow hole 101a is, for example, 0.2 mm to 1 mm. An interval between the flow holes 101a in a length direction of the holder 101 is, for example, 2 mm to 10 mm.

The flow holes 101a allow the cell suspension S to flow. The flow holes 101a are each formed to penetrate from an inner circumferential surface to an outer circumferential surface of the main portion 11.

The method of manufacturing the intravascular indwelling device of the embodiment is the same as the manufacturing method of the first embodiment except that the manufacturing jig 110 is used instead of the manufacturing jig 10 (refer to FIG. 2).

The holder 101 holding a stent 20 is inserted into the outer cylinder 2. The outer cylinder 2 accommodates the holder 1 in an internal space 2a.

A cell suspension S is introduced into the outer cylinder 2 (refer to FIG. 5). The manufacturing jig 110 is rotated around a support body 31 using a culture device 30 (refer to FIG. 6). The cell suspension S flows inside the outer cylinder 2. Some of the cell suspension S flows into the holder 101 through the flow hole 101a. Some of the cell suspension S flows out of the holder 101 through the flow hole 101a. In this way, when cells of interest are cultured while causing the cell suspension S to flow, the cells of interest are adhered to a wide range on a surface of the stent 20.

### Effects Obtained by Manufacturing Method of Second Embodiment

In the manufacturing method of the embodiment, since the holder 101 having the flow hole 101a is used, the cell suspension S inside the outer cylinder 2 can flow through the flow hole 101a of the holder 101. Therefore, it is possible to cause the cell suspension S inside the holder 101 to flow easily. Therefore, the cells of interest are easily in contact with the stent 20. Therefore, it is possible to cause the cells of interest to adhere to a wide range on the surface of the stent 20.

Since the holder 101 of the embodiment has the flow hole 101a, it is possible to cause the cell suspension S inside the holder 101 to flow easily. Therefore, it is possible to cause the cells of interest to adhere to a wide range on the surface of the stent 20.

### EXAMPLE

Effects of the invention will become apparent by Examples below. Further, the invention is not limited to the following Examples, and can be implemented with appropriate modifications within a range not changing the gist thereof.

### Example 1

The manufacturing jig 10 shown in FIG. 2 was manufactured. The holder 1 (an inner diameter of approximately 2.3 mm, and an outer diameter of main portion 11 of approximately 3 mm) is made of a fluorine-based polymer. The outer cylinder 2 (inner diameter of 6 mm) is a silicone tube. The stent 20 (refer to FIG. 7) is formed of a nickel-titanium alloy.

The holder 1 holding the stent 20 was accommodated in the outer cylinder 2, and the cell suspension S was supplied into the outer cylinder 2 (refer to FIG. 5). Late-outgrowth vascular endothelial progenitor cells (late EPC) were used as cells of interest.

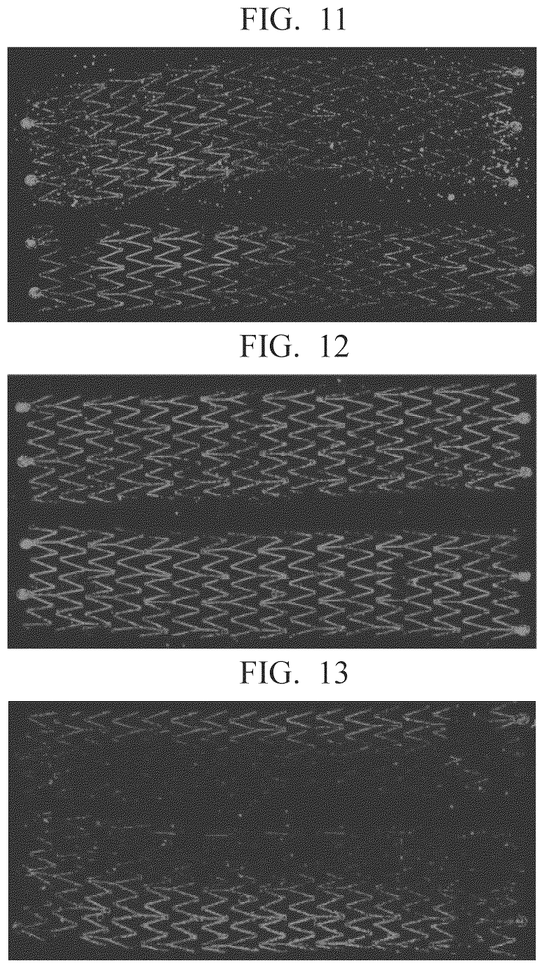

Using the culture device 30 shown in FIG. 6, cells of interest were cultured by performing a rotational operation of the manufacturing jig 10 such that the cell suspension S flows while being in contact with the stent 20. A culture temperature was 37° C. A culture period was 6 hours. The stent 20 was subjected to Hoechst staining. FIG. 11 is a photograph showing an inner surface of the stent divided into two along the central axis and expanded.

### Example 2

The manufacturing jig 110 shown in FIGS. 9 and 10 was manufactured. The holder 101 has the plurality of flow holes 101a (hole diameter of 0.5 mm). An interval between the flow holes 101a in the length direction of the holder 101 is approximately 10 mm. With other conditions the same as those in Example 1, cells of interest were cultured. The obtained stent 20 was subjected to Hoechst staining. FIG. 12 is a photograph showing an inner surface of the stent divided into two along the central axis and expanded.

### Comparative Example 1

A stent was held inside a plastic cylinder (inner diameter of 2.7 mm), and a cell suspension was supplied into the cylinder. Cells of interest were cultured by rotating the cylinder around an axis thereof. The obtained stent was subjected to Hoechst staining. FIG. 13 is a photograph showing an inner surface of the stent divided into two along the central axis and expanded.

As shown in FIGS. 11 to 13, the stents of Examples 1 and 2 (refer to FIGS. 11 and 12) showed adherence of cells of interest over a wider range compared to the stent of Comparative Example 1 (refer to FIG. 13). Particularly, adherence of the cells of interest was ascertained over a wide range in the stent of Example 2 (refer to FIG. 12).

### Example 3

Four manufacturing jigs 110 (refer to FIGS. 9 and 10) were prepared. For each of the manufacturing jigs 110, the holder 101 holding the stent 20 was accommodated in the outer cylinder 2, and the cell suspension S was supplied into the outer cylinder 2 (refer to FIG. 5). As the stent 20, a bare metal stent Cordis S.M.A.R.T. CONTROL Vascular Stent System (Cordis, C06040ML) (inner diameter of 2 mm) was used. The holder 101 (inner diameter of approximately 2.3 mm) is formed of a fluorine-based polymer. The outer cylinder 2 (inner diameter of 6 mm) is a silicone tube.

Late-outgrowth vascular endothelial progenitor cells (late EPC) were used as cells of interest. A culture medium used for the cell suspension S is Lonza, EGM™-2 Endothelial Cell Growth Medium-2 BulletKit™, CC-3162.

Concentrations of cells of interest in the cell suspension S supplied to the four manufacturing jigs 110 are as follows.
Condition A: 2 million cells/mL
Condition B: 1 million cells/mL
Condition C: 400,000 cells/mL
Condition D: 0 cell/mL

An amount of the cell suspension S supplied to each of the four manufacturing jigs 110 was 2 mL.

Four manufacturing jigs 110 were mounted on the culture device 30 shown in FIG. 6. Using the culture device 30, cells of interest were cultured by performing a rotational operation of the manufacturing jigs 110 such that the cell suspension S flows while being in contact with the stent 20. A culture temperature was 37° C. A culture period was 6 hours. The culture was performed in a 5% CO₂ environment. A rotation speed of the culture device 30 was set to 1 revolution per minute.

The cell suspension S was removed from the outer cylinder 2, and the stent 20 was washed three times with 2 mL of phosphate-buffered saline (PBS) (Gibco, 10010023).

A new culture medium (2 mL) was supplied into the outer cylinder 2. Using the culture device 30, culture was performed in a culture medium at a temperature of 37° C. for 6 hours in a 5% CO₂ environment. A rotation speed of the culture device 30 was set to one revolution per minute.

The culture medium in the outer cylinder 2 was collected, and the amount of lactic acid production and the amount of glucose consumption in the culture medium were measured using a blood analyzer (Abbott, i-STATI Analyzer) and a dedicated cartridge (lactic acid: i-STAT Cartridge CG4+, glucose: i-STAT Cartridge CHEM8+). The results are shown in Table 1.

The amount of lactic acid production was calculated as follows. Since the culture media obtained under conditions B and C were equal to or lower than a lactic acid measurement limit (0.30 mmol/L) of the i-STAT cartridge CG4+, the following calculation method was employed. An equal amount of lactic acid was added to the culture media obtained under conditions A to D, and an amount of lactic acid was measured in a state exceeding the measurement limit. An amount of lactic acid under condition D (corresponding to the amount of lactic acid originally contained in the culture medium and the amount of added lactic acid) was subtracted from the measured value. Using the obtained value, the amount of lactic acid production was calculated by converting the lactic acid value in the original culture medium, taking into account a change in liquid volume due to the addition of the lactic acid.

The amount of glucose consumption was calculated as follows. The amount of glucose consumption was calculated by subtracting the amount of glucose measured under each of the conditions A to C from the amount of glucose under condition D.

TABLE 1

| | Condition A | Condition B | Condition C |
|---|---|---|---|
| Lactic acid production amount | 0.51 | 0.22 | 0.04 |
| [mmol/L] | | | |
| Glucose consumption amount | 1.33 | 1.27 | 1.25 |
| [mmol/L] | | | |
| Ratio of lactic acid/glucose | 0.38 | 0.17 | 0.03 |
| (Q1/Q2) | | | |

From Table 1, it was ascertained that the ratio (Q1/Q2) between the lactic acid production amount Q1 and the glucose consumption amount Q2 was 0.1 or higher. Under condition A, it was ascertained that the ratio (Q1/Q2) was 0.3 or higher.

The stent 20 was subjected to Hoechst staining.

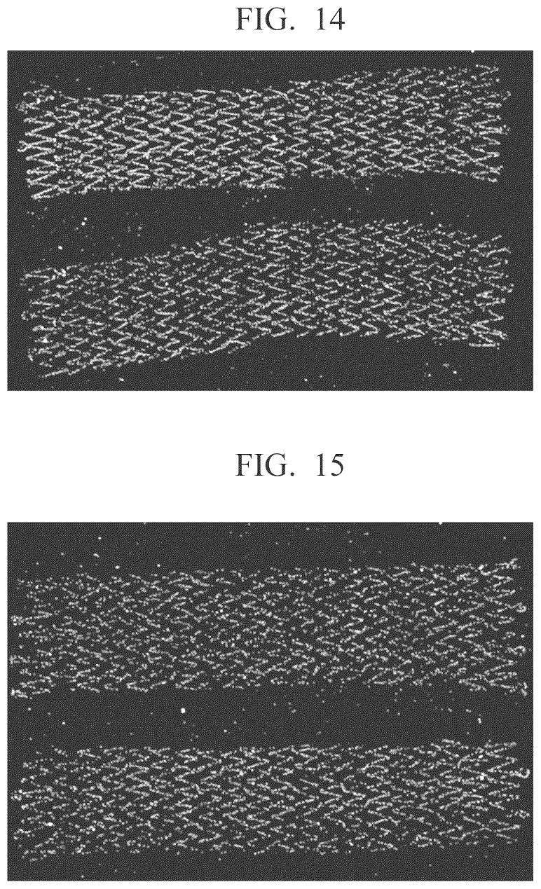

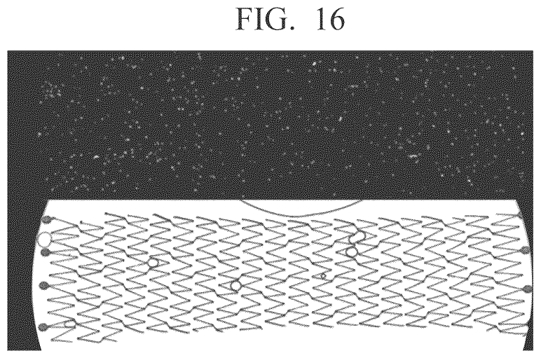

FIG. 14 is a photograph showing the inner surface of the stent 20 obtained under condition A. FIG. 15 is a photograph showing the inner surface of the stent 20 obtained under condition C. FIG. 16 is a photograph showing the inner surface of the stent 20 obtained under condition D. In FIGS. 14 to 16, the stent 20 was divided into two along the central axis and expanded. An upper image in FIG. 16 is an image of Hoechst staining. A lower image in FIG. 16 is a bright-field image.

As shown in FIGS. 14 and 15, a larger number of cells of interest were observed to adhere to the stents 20 under conditions A and C than to the stent 20 (refer to FIG. 16) under conditions D. Particularly, the stent 20 under condition A (refer to FIG. 14) was observed to have dense cells of interest along surfaces of wires (linear objects) and was ascertained that a larger amount of cells of interest were adhered even when compared to the stent 20 under condition C (refer to FIG. 15).

Although embodiments of the invention have been described above, configurations, combinations thereof, or the like in the embodiments are examples, and additions, omissions, substitutions, and other changes to the configurations can be made within a range not departing from the gist of the invention. The invention is not limited by the embodiments.

For example, in the embodiment described above, the manufacturing jig 10 is rotated around a horizontal axis using the culture device 30, but an operation of the manufacturing jig 10 is not particularly limited as long as it is possible to realize a flow of the cell suspension S to the stent 20. For example, the manufacturing jig 10 may be caused to perform a reciprocating operation linearly in a length direction or a radial direction. An inclination of the manufacturing jig 10 may be repeatedly changed. The manufacturing jig 10 may be caused to perform a rotational operation around an axis extending in a vertical direction.

### REFERENCE SIGNS LIST

1 Holder
1a Internal space
2 Outer cylinder
5 Check valve (backflow prevention structure)
20 Stent (intravascular indwelling device)
23 Cell of interest
101 Holder (intravascular-indwelling-device manufacturing holder)
101a Flow hole
S Cell suspension (suspension)

1. A method of manufacturing an intravascular indwelling device having cells of interest adhered thereto, which are at least one of vascular endothelial cells and vascular endothelial progenitor cells,
the method comprising:
using a cylindrical holder holding the intravascular indwelling device in an internal space, and an outer cylinder having an inner diameter larger than an outer diameter of the holder; and
accommodating the holder holding the intravascular indwelling device in the outer cylinder, supplying a suspension of the cells of interest into the outer cylinder, culturing the cells of interest by operating the outer cylinder and the holder such that the suspension flows to the intravascular indwelling device while being in contact with the intravascular indwelling device.

2. The method of manufacturing an intravascular indwelling device according to claim 1, wherein
the holder has one or more flow holes allowing the suspension to flow therethrough and are formed to penetrate from an inner circumferential surface to an outer circumferential surface of the holder.

3. The method of manufacturing an intravascular indwelling device according to claim 1, further comprising
a backflow prevention structure restricting an inflow of a gas into the outer cylinder and is provided at least on one end part of the outer cylinder.

4. The method of manufacturing an intravascular indwelling device according to claim 1, further comprising
causing the outer cylinder and the holder to rotate around an axis parallel to a central axis of the outer cylinder when the suspension flows.

5. An intravascular-indwelling-device manufacturing holder used for manufacturing an intravascular indwelling device having cells of interest adhered thereto, which are at least one of vascular endothelial cells and vascular endothelial progenitor cells,
the intravascular-indwelling-device manufacturing holder
having a cylindrical shape capable of holding the intravascular indwelling device in an internal space, and
having one or more flow holes allowing the suspension of the cells of interest to flow therethrough and are formed to penetrate from an inner circumferential surface to an outer circumferential surface.

6. A method of evaluating an intravascular indwelling device having cells of interest adhered thereto, which are at least one of vascular endothelial cells and vascular endothelial progenitor cells,
the method comprising
ascertaining that a ratio (Q1/Q2) of a lactic acid production amount Q1 to a glucose consumption amount Q2 of the cells of interest is 0.1 or higher when the intravascular indwelling device is cultured in a culture medium at a temperature of 37° C. for 6 hours in a 5% CO2 environment.

7. The method of evaluating an intravascular indwelling device according to claim 6, wherein
the method ascertains that the ratio (Q1/Q2) is 0 3 or higher.
